(19) United States
(12) Patent Application Publication
(10) Pub. No.: US 2025/0213668 A1
(43) Pub. Date: Jul. 3, 2025

(54) Use of Bordetella Strains for the Treatment of Chronic Obstructive Pulmonary Disease

(71) Applicants: Institut National de la Santé et de la Recherche Médicale, Paris (FR); Centre National de la Recherche Scientifique, Paris (FR); Centre Hospitalier Universitaire de Lille, Lille (FR); Institut Pasteur de Lille, Lille (FR); Université de Lille, Lille (FR)

(72) Inventors: Camille Locht, Brussels (BE); Philippe Gosset, Lille (FR); Muriel Pichavant, Lille (FR)

(21) Appl. No.: 19/028,179

(22) Filed: Jan. 17, 2025

Related U.S. Application Data
(63) Continuation of application No. PCT/EP2023/070301, filed on Jul. 21, 2023.

(30) Foreign Application Priority Data
Jul. 22, 2022 (EP) 22306105.2

(51) Int. Cl. A61K 39/02; A61K 39/00; A61P 11/00

(52) U.S. Cl. CPC A61K 39/099; A61P 11/00; A61K 2039/522

(57) ABSTRACT

Chronic obstructive pulmonary disease is a major clinical challenge mostly due to cigarette smoke exposure and affects more than 200 million people. The inventors tested if exposure to the Bordetella pertussis BPZE1 strain could modulate outcomes of chronic exposure to cigarette smoke in mice. In particular, they showed in mice chronically exposed to cigarette smoke that preventive and/or curative vaccination using BPZE1 could limit the lung inflammation and strongly contribute to the prevention of lung function decline. BPZE1 vaccination modulated pulmonary antigen presenting cells (macrophages and dendritic cells) to switch the immune response, by decreasing the IL-17 inflammatory pathway involved in the pathology of COPD itself, and by favouring a tolerogenic response (IL-10). Together, the data show that vaccination with BPZE1 of mice chronically exposed to cigarette smoke limits the development of chronic obstructive pulmonary disease outcomes and thus represents an interesting therapy.

### CROSS-REFERENCE TO RELATED APPLICATIONS

The present application is a by-pass continuation under 35 U.S.C. 111 (a) of international patent application number PCT/EP2023/070301 filed on Jul. 21, 2023 which claims the priority of EP patent application Ser. No. 22/306,105.2 filed on Jul. 22, 2022.

### STATEMENT AS TO FEDERALLY SPONSORED RESEARCH

Not applicable.

### FIELD OF THE INVENTION

The present invention is in the field of medicine, in particular immunology and pneumology.

### BACKGROUND

Chronic obstructive pulmonary disease (COPD) is a leading cause of morbidity and mortality worldwide. This disease affects more than 200 million people and is the fourth leading cause of death. Cigarette smoke (CS) exposure is the most important risk factor for COPD and smoking cessation is the only intervention that leads to a decrease in the rate of lung function decline. COPD is defined as a preventable disease state characterized by airway inflammation, airflow limitation that is not reversible. Neutrophils and macrophages are the most important inflammatory cells participating in the pathophysiology of COPD with airway epithelial cells. The migration and the activation of these cells involve Th17 cytokines mostly released by CD4+T lymphocytes (Hong S C, Lee S H. Role of th17 cell and autoimmunity in chronic obstructive pulmonary disease. Immune Netw. 2010 August; 10 (4): 109-14. doi: 10.4110/in.2010.10.4.109. Epub 2010 Aug. 31). Indeed, IL-17A, IL-17F and IL-22 act as inducers for CXCL8, CXCL1, CXCL5, G-CSF, and GM-CSF secretion by airway epithelial cells and macrophages that subsequently trigger differentiation, proliferation and recruitment of neutrophils (Aujla S J, Dubin P J, Kolls J K. Interleukin-17 in pulmonary host defense. Exp Lung Res. 2007; 33 (10): 507-518). This leads to the secretion of proteases (elastase, metalloelastase, MMPs) that are responsible for airway remodeling and alveolar wall destruction called emphysema.

BPZE1 is a live attenuated pertussis vaccine. When delivered nasally as a single drop, was found to fully protect against Bordetella pertussis challenge in pre-clinical models for at least up to one year. It was also shown to be safe, even in severely immune-compromised mice and genetically stable after at least one year of continuous passages in vivo. BPZE1 has successfully undergone several clinical trials and was found to be safe in humans, able to transiently colonize the respiratory tract and to induce immune responses in all colonized individuals to all antigens tested. It was demonstrated that BPZE1 promotes human dendritic cell CCL21-induced migration and drives a Th1/Th17 response (Schiavoni I, Fedele G, Quattrini A, Bianco M, Schnoeller C, Openshaw P J, Locht C, Ausiello C M. Live attenuated B. pertussis BPZE1 rescues the immune functions of Respiratory Syncytial virus infected human dendritic cells by promoting Th1/Th17 responses. PLoS One. 2014 Jun. 26; 9 (6):e100166). In pre-clinical studies, the vaccine was found to have interesting anti-inflammatory properties, without being immunosuppressive. For instance, BPZE1 was also found to protect against inflammation resulting from heterologous airway infections, including those caused by other Bordetella species, influenza virus and respiratory syncytial virus (Li R, Lim A, Phoon M C, Narasaraju T, Ng J K, Poh W P, Sim M K, Chow V T, Locht C, Alonso S. Attenuated Bordetella pertussis protects against highly pathogenic influenza A viruses by dampening the cytokine storm. J Virol. 2010 July; 84 (14): 7105-13; Schiavoni I, Fedele G, Quattrini A, Bianco M, Schnoeller C, Openshaw P J, Locht C, Ausiello C M. Live attenuated B. pertussis BPZE1 rescues the immune functions of Respiratory Syncytial virus infected human dendritic cells by promoting Th1/Th17 responses. PLoS One. 2014 Jun. 26; 9 (6):e100166). Furthermore, the heterologous protection conferred by BPZE1 was also observed for non-infectious inflammatory diseases, such as allergic asthma, as well as for inflammatory disorders outside of the respiratory tract, such as contact dermatitis (Li R, Cheng C, Chong S Z, Lim A R, Goh Y F, Locht C, Kemeny D M, Angeli V, Wong W S, Alonso S. Attenuated Bordetella pertussis BPZE1 protects against allergic airway inflammation and contact dermatitis in mouse models. Allergy. 2012 October; 67 (10): 1250-8). Some of these protective effects have been further investigated and were found to rely on the ability of BPZE1 to induce Th17 responses (Schnoeller C, Roux X, Sawant D, Raze D, Olszewska W, Locht C, Openshaw P J. Attenuated Bordetella pertussis vaccine protects against respiratory syncytial virus disease via an IL-17-dependent mechanism. Am J Respir Crit Care Med. 2014 January; 189 (2): 194-202).

Despite the fact that asthma and COPD are both characterized by airway obstruction, there are marked differences in the pattern of airway inflammation, with different inflammatory cells recruited, different mediators produced, distinct tissue lesions (with subepithelial fibrosis, bronchial metaplasia and emphysema characterizing COPD) and differing responses to therapy (Barnes, P. J. “Similarities and differences in inflammatory mechanisms of asthma and COPD.” Breathe 7.3 (2011): 229-238; Cukic V, Lovre V, Dragisic D, Ustamujic A. Asthma and Chronic Obstructive Pulmonary Disease (COPD)—Differences and Similarities. Mater Sociomed. 2012; 24 (2): 100-5). For instance, the inflammation seen in asthma is mainly located in the larger conducting airways, although small airways may also be involved in more severe disease, but the lung parenchyma is not affected. By contrast, COPD predominantly affects the small airways and lung parenchyma, although similar inflammatory changes may also be found in larger airways (Jeffery P K. Comparison of the structural and inflammatory features of COPD and asthma. Chest 2000; 117: 251S-260S). The differences in inflammation between asthma and COPD are also linked to differences in the immunological mechanisms of these two diseases. In asthmatic patients, there is an increase in the number of CD4+ T cells in the airways and these are predominantly Th2 cells (Meyer E H, DeKruyff R H, Umetsu D T. T Cells and NKT Cells in the Pathogenesis of Asthma. Annu Rev Med 2008; 59:281-292). In contrast to asthma, the CD4+ T cells that accumulate the airways and lungs of patients with COPD are mainly Th-1 cells and is also driven by a Th17 response (see supra). Therefore, the use of BPZE1 that increases the Th1/Th17 response (see supra) is thus counter-intuitive for decreasing inflammation in COPD.

To summarize, asthma and COPD have different etiology, different symptoms, different types of airway inflammation, different inflammatory cells, different mediators, different lung lesions, different response to therapy, and different course (Cukic V, Lovre V, Dragisic D, Ustamujic A. Asthma and Chronic Obstructive Pulmonary Disease (COPD)—Differences and Similarities. Mater Sociomed. 2012; 24 (2): 100-5). Therefore, any teaching that results from the study of asthma does not turn straightly to the application of the same teaching for COPD. With said consideration in mind, the interest of BPZE1 for the treatment of COPD was not predictable from the prior art.

### SUMMARY

The present invention is defined by the claims. In particular, the present invention relates to use of Bordetella strains for the treatment of chronic obstructive pulmonary disease (COPD).

### DESCRIPTION OF THE DRAWINGS

FIG. 1A is a timeline illustrating a protocol of an experiment designed to analyze the effects of BPZE-1 vaccination on mice chronically exposed to cigarette smoke. Mice were daily exposed to cigarette smoke (CS) as followed 5 cigarettes/day, 5 days/week over a period of 12 weeks to develop symptoms associated with COPD or exposed to air (Air). Mice were vaccinated by intranasal exposure to BPZE-1 either before (T1: 2 weeks before), either during (T2: 6 weeks after the beginning) the course of chronic exposure to CS or both, before and during CS exposure (T1+T2). FIG. 1B is a series of two graphs showing the cell counts in the bronchoalveolar lavages (BAL) and in lung tissues in mice subjected to the experiment referred to in FIG. 1A.

FIGS. 2A and 2B are a series of graphs showing the effects of BPZE-1 vaccination during exposure to cigarette smoke (T2) on lung function. Mice were daily exposed to cigarette smoke (CS) for 12 weeks and vaccinated by BPZE-1 6 weeks after the beginning of CS exposure (CS+BPZE). FIG. 2A is a series of three graphs showing the results of this experiment on tissue damping (G) and tissue elasticity (H) (measured byflexiVent®), and tissue hysteresivity (ratio G/H). FIG. 2B is a series of two graphs showing the results of this experiment on inspiratory capacity (IC) and static compliance (Cst) as measured byflexiVent®. *, p<0.05; **, p<0.01; ***, p<0.005.

FIGS. 3A and 3B are a series of graphs showing the effects of BPZE-1 vaccination during exposure to cigarette smoke (T2) on lung inflammation. Mice were daily exposed to cigarette smoke (CS) for 12 weeks (CS) and vaccinated by BPZE-1 6 weeks after the beginning of CS exposure (CS+BPZE). Control mice were exposed to air (Air). FIG. 3A shows the levels of inflammatory cytokines including IL-6, KC, IL-17 and IL-22 that were measured in bronchoalveolar lavages (BAL). FIG. 3B shows the level of this cytokines in lung tissue lysates (B). *, p<0.05; **, p<0.01; ***, p<0.005.

FIGS. 4A and 4B are a series of graphs showing the effects of BPZE-1 vaccination during exposure to cigarette smoke (T2) on lung inflammation parameters. Mice were daily exposed to cigarette smoke for 12 weeks (CS) or air (Air) and vaccinated by BPZE-1 6 weeks after the beginning of CS (CS+T2; CS+BPZE1) or air (Air+T2; Air+BPZE1) exposure. FIG. 4A shows the levels of IL-23 and IL-10 mRNA in total lung tissues. FIG. 4B shows the levels of mRNA coding for RAGE and AhR in enriched lung tissue extracts. *, p<0.05.

FIG. 5 is a series of graphs showing the effects of BPZE-1 vaccination during exposure to cigarette smoke (T2) on lung immune cell recruitment and activation. Mice were daily exposed to cigarette smoke for 12 weeks (CS) and vaccinated by BPZE-1 6 weeks after the beginning of CS exposure (CS+BPZE). Control mice were exposed to air (Air). Immunophenotyping of cells infiltrating lung tissue was performed by flow cytometry. *, p<0.05; **, p<0.01; ***, p<0.005.

FIGS. 6A and 6B are a series of graphs showing the effects of BPZE-1 vaccination during exposure to cigarette smoke (T2) on antigen presenting cells. Mice were daily exposed to cigarette smoke (CS) or air (Air) for 12 weeks and vaccinated by BPZE-1 6 weeks after the beginning of CS exposure (CS+BPZE1; Air+BPZE1). Pulmonary antigen presenting cells, including alveolar macrophages, inflammatory monocytes, and CD11b+ and CD103+ dendritic cells, were sorted by flow cytometry. Levels of IL-6, IL-23 and IL-10 mRNA were measured and are expressed as fold increases compared to Air-exposed mice. FIG. 6A shows the results from alveolar macrophages and inflammatory monocytes. FIG. 6B shows the results CD11b+ dendritic cells and CD103+ dendritic cells.

### DETAILED DESCRIPTION

Chronic obstructive pulmonary disease is a major clinical challenge mostly due to cigarette smoke exposure and affects more than 200 million people. Safety and efficacy of treatments or preventive interventions against such chronic diseases themselves have shown their limits, despite intensive research and development efforts deployed in this area. Therapeutic interventions remain to be found. The inventors tested if exposure to BPZE1 could modulate outcomes of chronic exposure to cigarette smoke in mice. In particular, they showed in mice chronically exposed to cigarette smoke that preventive and/or curative vaccination using BPZE1 could limit the lung inflammation and strongly contribute to the prevention of lung function decline. BPZE1 vaccination modulated pulmonary antigen presenting cells (macrophages and dendritic cells) to switch the immune response, by surprisingly decreasing the IL-17 inflammatory pathway involved in the pathology of COPD itself, and by favouring a tolerogenic response (IL-10). Together, the data show that vaccination with BPZE1 of mice chronically exposed to cigarette smoke limits the development of chronic obstructive pulmonary disease outcomes and thus represents an interesting therapy.

### Main Definitions

As used herein, the term “chronic obstructive pulmonary disease” or “COPD” has its general meaning in the art and refers to a set of physiological symptoms including chronic cough, expectoration, exertional dyspnea and a significant, progressive reduction in airflow that may or may not be partly reversible. COPD is a disease characterized by a progressive airflow limitation caused by an abnormal inflammatory reaction to the chronic inhalation of particles. The Global Initiative for Chronic Obstructive Lung Disease (GOLD) has classified 4 different stages of COPD (Table A).

TABLE A
Gold classification: The volume in a one-second forced exhalation is called
the forced expiratory volume in one second (FEV1), measured in liters. The
total exhaled breath is called the forced vital capacity (FVC), also measured
in liters. In people with normal lung function, FEV1 is at least 70% of FVC.

| | | | |
|---|---|---|---|
| Stage I | Mild COPD | FEV1/FVC < 0.70 | FEV1 ≥80% normal |
| Stage II | Moderate COPD | FEV1/FVC < 0.70 | FEV1 50-79% normal |
| Stage III | Severe COPD | FEV1/FVC < 0.70 | FEV1 30-49% normal |
| Stage IV | Very Severe COPD | FEV1/FVC < 0.70 | FEV1 <30% normal, |
| | | | or <50% normal with chronic |
| | | | respiratory failure present |

As used herein, the term “Bordetella strain” includes strains from Bordetella pertussis, Bordetella parapertussis, and Bordetella bronchiseptica.

As used herein, the term “PTX” refers to pertussis toxin, an ADP-ribosylating toxin synthesized and secreted by Bordetella pertussis. PTX is comprised of five different subunits (named S1-S5) with each complex containing two copies of S4. The subunits are arranged in an A-B structure. The A component is enzymatically active and is formed by the S1 subunit, while the B component is the receptor-binding portion and is made up of subunits S2-S5. As used herein, the term “DNT” refers to pertussis dermonecrotic toxin, which is a heat labile toxin that can induce localized lesions in mice and other laboratory animals when it is injected intradermally.

As used herein, the term “TCT” refers to tracheal cytotoxin, which is a virulence factor synthesized by Bordetellae. TCT is a peptidoglycan fragment and has the ability to induce interleukin-1 production and nitric oxide synthase. It has the ability to cause stasis of cilia and has lethal effects on respiratory epithelial cells.

As used herein, the term “ampG” refers to a gene that codes for a permease for the transport of 1,6-GlcNac-anhydro-MurNAc.

As used herein, the term “pertactin” refers to the outer surface membrane protein produced by Bordetella pertussis and its close relatives, such as Bordetella parapertussis, and may be involved in the binding of Bordetella bacteria to host cells as described in Leininger et al., Proc. Natl. Acad. Sci. USA, 1991, 88:345-9.

As used herein, the term “attenuated” refers to a weakened, less virulent Bordetella strain that is capable of stimulating an immune response and creating protective immunity, but does not generally cause illness.

As used herein, the term “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is an amount that is effective to ameliorate a symptom of a disease. A therapeutically effective amount can be a “prophylactically effective amount” as prophylaxis can be considered therapy.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein, the terms “live vaccine composition”, “live vaccine”, “live bacterial vaccine”, and similar terms refer to a composition comprising a strain of live Bordetella bacteria that provides at least partial protective immunity against a disease, condition, or disorder.

As used herein, the term “adjuvant” refers to a compound that can induce and/or enhance the immune response against an antigen when administered to a patient or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dendritic cells (DCs).

As used herein, the term “nasal administration” refers to any form of administration whereby an active ingredient is propelled or otherwise introduced into the nasal passages of a patient so that it contacts the respiratory epithelium of the nasal cavity, from which it is absorbed into the systemic circulation. Nasal administration can also involve contacting the olfactory epithelium, which is located at the top of the nasal cavity between the central nasal septum and the lateral wall of each main nasal passage. The region of the nasal cavity immediately surrounding the olfactory epithelium is free of airflow. Thus, specialized methods must typically be employed to achieve significant absorption across the olfactory epithelium.

As used herein, the term “aerosol” is used in its conventional sense as referring to very fine liquid or solid particles carried by a propellant gas under pressure to a site of therapeutic application. A pharmaceutical aerosol can contain a therapeutically active compound, which can be dissolved, suspended, or emulsified in a mixture of a fluid carrier and a propellant. The aerosol can be in the form of a solution, suspension, emulsion, powder, or semi-solid preparation. Aerosols are intended for administration as fine, solid particles or as liquid mists via the respiratory tract of a patient. Various types of propellants can be utilized including, but not limited to, hydrocarbons or other suitable gases. Aerosols can also be delivered with a nebulizer, which generates very fine liquid particles of substantially uniform size within a gas. A liquid containing the active compound is dispersed as droplets, which can be carried by a current of air out of the nebulizer and into the respiratory tract of the patient.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patents, and patent applications mentioned herein are incorporated by reference in their entirety. In the case of conflict, the present specification, including definitions will control. In addition, the particular embodiments discussed below are illustrative only and not intended to be limiting.

### Methods of the Present Invention

Accordingly, the first object of the present invention relates to a method of treating chronic obstructive pulmonary disease in a patient in need thereof comprising administering a therapeutically effective amount of a mutated Bordetella strain, wherein the strain comprises a mutated pertussis toxin (ptx) gene, a deleted or mutated dermonecrotic (dnt) gene, and a heterologous ampG gene.

In some embodiments, the patient suffers from moderate COPD. In some embodiments, the patient suffers from a severe or very severe COPD.

In some embodiments, the method of the present invention is particularly suitable for preventing progression of COPD in a patient. More particularly, the method of the present invention is particularly suitable for preventing progression of COPD in a patient from one stage to a subsequent stage in the GOLD classification.

According to the present invention, the mutated Bordetella strain of the present invention is particularly suitable for raising an immune response so as to protect the patient against COPD or against its consequences/symptoms. More particularly, the mutated Bordetella strain of the present invention modulates pulmonary antigen presenting cells (macrophages and dendritic cells) to switch the immune response, by decreasing the IL-17 inflammatory pathway involved in the pathology of COPD itself, and by favouring a tolerogenic response (IL-10).

In some embodiments, the Bordetella starting strain which is mutated can be any Bordetella strain including Bordetella pertussis, Bordetella parapertussis, and Bordetella bronchiseptica. In some embodiments the starting strain used to obtain the mutated Bordetella strain is Bordetella pertussis.

Typically, the construction of the mutated Bordetella strain can begin with replacing the Bordetella ampG gene in the strain with a heterologous ampG gene. Any heterologous ampG gene known in the art can be used. Examples of these can include all gram-negative bacteria that release very small amounts of peptidoglycan fragments into the medium per generation. Examples of gram-negative bacteria include, but are not limited to: Escherichia coli, Salmonella, Enterobacteriaceae, Pseudomonas, Moraxella, Helicobacter, Stenotrophomonas, Legionella, and the like. Typically, by replacing the Bordetella ampG gene with a heterologous ampG gene, the amount of tracheal cytotoxin (TCT) produced in the resulting strain expresses less than 1% residual TCT activity. In some embodiments, the amount of TCT toxin expressed by the resulting strain is between about 0.6% to 1% residual TCT activity or about 0.4% to 3% residual TCT activity or about 0.3% to 5% residual TCT activity.

PTX is a major virulence factor responsible for the systemic effects of B. pertussis infections, as well as one of the major protective antigens. Due to its properties, the natural ptx gene can be replaced by a mutated version so that the enzymatically active moiety S1 codes for an enzymatically inactive toxin, but the immunogenic properties of the pertussis toxin are not affected. This can be accomplished by replacing the arginine (Arg) at position 9 of the sequence with lysine (Lys) (R9K). Furthermore, a glutamic acid (Glu) at position 129 can be replaced with a glycine (Gly) (E129G). Generally, these amino acid positions are involved in substrate binding and catalysis, respectively. In some embodiments, other mutations can also be made such as those described in U.S. Pat. No. 6,713,072, incorporated herein by reference, as well as any known or other mutations able to reduce the toxin activity. In some embodiments, allelic exchange can first be used to delete the ptx operon and then to insert a mutated version.

In some embodiments, the dnt gene can be removed from the Bordetella strain using allelic exchange. Besides the total removal, the enzymatic activity can also be inhibited by a point mutation. Since DNT is constituted by a receptor-binding domain in the N-terminal region and a catalytic domain in the C-terminal part, a point mutation in the dnt gene to replace Cys-1305 to Ala-1305 inhibits the enzyme activity of DNT (Kashimoto T., Katahira J, Cornejo W R, Masuda M, Fukuoh A, Matsuzawa T, Ohnishi T, Horiguchi Y. (1999) Identification of functional domains of Bordetella dermonecroting toxin. Infect. Immun. 67:3727-32).

Besides allelic exchange to insert the mutated ptx gene and the inhibited or deleted dnt gene, the open reading frame of a gene can be interrupted by insertion of a genetic sequence or plasmid. This method is also contemplated. Other methods of generating mutant strains are generally well known in the art.

In some embodiments, the mutated Bordetella strain is BPZE1. The BPZE1 strain has been deposited with the Collection Nationale de Cultures de Microorganismes (CNCM) in Paris, France under the Budapest Treaty on Mar. 9, 2006 and assigned the number CNCM I-3585. The mutations introduced into BPZE1 generally result in attenuation but also allow the bacteria to colonize and persist. Thus, in some embodiments, BPZE1 can induce mucosal immunity and systemic immunity when administered to a patient in need thereof. Thus, in some embodiments, the Bordetella strain is identified by accession number CNCM I-3585.

According to the present invention, the strain is a triple mutant Bordetella strain. However, the strains that can be used are not limited to only the mutants described herein. Other additional mutations can be undertaken such as pertactin deficient mutants, adenylate cyclase (AC) deficient mutants, filamentous hemagglutinin (FHA), and any of the bvg-regulated components.

In some embodiments, the mutated Bordetella strain of the present invention is also deficient for pertactin. As used herein, a “pertactin-deficient” Bordetella strain is one that exhibits at least less than 50% (e.g., less than 50, 40, 30, 20, 10, 5, 4, 3, 2, or 1%) of the pertactin activity found in BPZE1 under the conditions described in WO2017167834, one that exhibits no detectable pertactin activity, or one that exhibits not detectable expression of pertactin as determined by Western blotting. Typically, the pertactin-deficient Bordetella strain is obtained as described in WO2017167834.

In some embodiments, the mutated Bordetella strain of the present invention is attenuated. More particularly chemically- or heat killed Bordetella strains are used.

In some embodiments, the mutated Bordetella strains is administered to the patient as pharmaceutical compositions, more particularly vaccine compositions. Typically, the composition can comprise, in addition to one or more of the strains, a pharmaceutically acceptable excipient, carrier, buffer, stabilizer, or other materials well known to those skilled in the art. Such materials should typically be non-toxic and should not typically interfere with the efficacy of the active ingredient. The precise nature of the carrier or other material can depend on the route of administration, e.g., oral, intravenous, cutaneous or subcutaneous, nasal, intramuscular, or intraperitoneal routes.

In some embodiments, the mutated Bordetella strain is administered to the patient as live vaccine.

In some embodiments, the mutated Bordetella strain is administered to the patient by nasal administration. In some embodiments, the composition is administered via the nose of the patient, e.g., intranasally or via inhalation. In some embodiments, the mutated Bordetella strain of the present invention is administered to the patient by an aerosol.

The actual amount administered, and rate and time-course of administration, will depend on the nature and severity of COPD. Prescription of treatment, e.g., decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in the latest edition of Remington's Pharmaceutical Science, Mack Publishing Company, Easton, PA (“Remington's”). Typically, the composition can be administered alone or in combination with other treatments, either simultaneously or sequentially dependent upon the condition to be treated. In some embodiments, a composition is administered in one dose to a patient. In some embodiments, a composition is administered in more than one dose, e.g., two doses. In some embodiments, a composition is administered in 1, 2, 3, 4, or greater than 4 doses. The number of doses can vary as needed, for example the number of doses administered to a mammal can be 1, 2, 3, 4, 5, 6, 7, 8, 9, 10, or more doses. In some embodiments, the method for treating COPD, includes administering to a patient in need thereof a first vaccine composition (comprising e.g., BPZE1) followed by a second vaccine composition administration (comprising e.g., BPZE1). Typically, the time range between each dose of the composition can be about 1-6 days, or about 1, 2, 3, 4, 5, 6, 7, 8, 9, 10, 11, 12, 13, 14, 15, 20, 30, 40, 50, 60, 70, 80, 90, or more weeks. In some embodiments, the time range between each dose is about 3 weeks. In some embodiments, prime-boost-style methods can be employed where a composition can be delivered in a “priming” step and, subsequently, a composition is delivered in a “boosting” step.

The composition can be administered in conjunction with other immunoregulatory agents, including adjuvants. In particular, the adjuvant is selected from the group consisting of mineral salts, such as aluminium salts and calcium salts. The adjuvants include mineral salts such as hydroxides (e.g., oxyhydroxides), phosphates (e.g., hydroxyphosphates, orthophosphates), sulfates, and the like (e.g., see chapters 8 & 9 of Vaccine Design . . . (1995) eds. Powell & Newman. ISBN: 030644867X. Plenum.), or mixtures of different mineral compounds (e.g., a mixture of a phosphate and a hydroxide adjuvant, optionally with an excess of the phosphate), with the compounds taking any suitable form (e.g., gel, crystalline, amorphous, and the like), and with adsorption to the salt(s) being contemplated. The mineral containing compositions can also be formulated as a particle of metal salt (WO/0023105). Oil-emulsion compositions suitable for use as adjuvants can include squalene-water emulsions, such as MF59 (5% Squalene, 0.5% Tween® 80, and 0.5% Span® 85, formulated into submicron particles using a microfluidizer). See, e.g., WO90/14837. See also, Podda, “The adjuvanted influenza vaccines with novel adjuvants: experience with the MF59-adjuvanted vaccine”, Vaccine 19:2673-2680, 2001. In other related aspects, adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween® 80 (polyoxyelthylenesorbitan monooleate), and/or 0.25-1.0% Span® 85 (sorbitan trioleate), and, optionally, N-acetylmuramyl-L-alanyl-D-isogluatminyl-L-alanine-2-(1′-2′-dipalmitoyl-s-n-glycero-3-hydroxyphosphoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). Saponin formulations, can also be used as adjuvants. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21 DesertKing®, as well as lipid formulations, such as Immunostimulating Complexs (ISCOMs; see below). Adjuvants can include bacterial or microbial derivatives such as: non-toxic derivatives of enterobacterial lipopolysaccharide (LPS), lipid A derivatives (e.g, derivatives of lipid A from Escherichia coli such as OM-174), immunostimulatory oligonucleotides (e.g. nucleotide sequences containing a CpG motif), and A DP-ribosylating toxins and detoxified derivatives thereof. Liposomes also can be used as adjuvant. Examples of liposome formulations suitable for use as adjuvants are described in U.S. Pat. Nos. 6,090,406, 5,916,588, and EP 0 626 169. Adjuvants can also include polyoxyethylene ethers and polyoxyethylene esters. WO99/52549. Such formulations can further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152). Human immunomodulators suitable for use as adjuvants can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

The invention will be further illustrated by the following figures and examples. However, these examples and figures should not be interpreted in any way as limiting the scope of the present invention.

### Examples

### Methods

### Reagents and Abs

mAbs against mouse CD3 (APC-conjugated), CD5 (FITC-conjugated), NK1.1 (PerCp-Cy5.5-conjugated), TCR-β (V450-conjugated), CD25 (APC-conjugated), CD69 (Alexa700-conjugated), CD11b (V450-conjugated), Ly-6G (APC-Cy7-conjugated), CD8 (V500-conjugated), CD4 (APC-conjugated), CD103 (PE-conjugated), CD11c (APC-conjugated), CD45 (Q-dot605-conjugated), F4/80 (PerCP-Cy5.5-conjugated), CD86 (PE-conjugated), I-Ab (FITC-conjugated), and isotype controls were purchased from Biolegend (Le Pont de Claix, France). PE-conjugated PBS57-loaded CD1d tetramer was from the National Institute of Allergy and Infectious Diseases Tetramer Facility (Emory University, Atlanta, GA). 1R6F research cigarettes were purchased from University of Kentucky.

### Mice

Six- to eight-week-old male wild-type (WT) C57BL/6 (H-2 Db) mice were purchased from Janvier (Le Genest-St-Isle, France). For CS exposure, mice were maintained in the Animal Resource Center at Pasteur Institute, Lille (Lille, France). All animal work conformed to the guidelines of Animal Care and Use Committee from Nord Pas-De-Calais (agreement no. AF 16/20090).

### Cigarette Smoke Exposure

Mice were placed in the inhalation chamber inside an exhaustion chapel (Emka, Scireq, Canada) and exposed to CS generated from 5 cigarettes per day, 5 days a week, and up to 12 weeks. The negative-control group was exposed to ambient air. Research cigarettes 1R6F were obtained from the University of Kentucky Tobacco and Health Research Institute (Lexington, KY, USA).

### Measurement of Lung Function

Lung function was assessed by invasive measurement of airway resistance, in which anesthetized and tracheotomized mice were mechanically ventilated (Pichavant M et al., Mucosal Immunol 2014). We computed tissue damping (G), tissue elasticity (H), Inspiratory capacity (IC), and the static compliance (Cst) by fitting flow, volume and pressure to an equation of motion (flexiVent® System, Scireq, Canada). We also calculated the tissue hysteresivity as the ratio G/H.

### Assessment of Airway Inflammation and Remodeling

Mice were sacrificed at the end of the protocol for sampling the lung lumen by bronchoalveolar lavage (BAL) as well as the lung tissue. Lungs were perfused with PBS, excised and finely minced, followed by enzymatic digestion for 20 min at 37° C. in RPMI 1640 containing 1 mg/ml collagenase type VIII (Sigma Aldrich) and 1 μg/ml DNase type I (Sigma Aldrich). After wash, lung homogenates were centrifuged in a 30% Percoll gradient. The pelleted cells were washed and red blood cells were removed with lysis buffer (Sigma Lysis). Pulmonary immune cells were characterized by flow cytometry.

### Pulmonary APC Cell Sorting

Pulmonary APC were purified from the lungs of naive animals and mice exposed to CS 2 weeks after BPZE1 vaccination, on the basis of F4/80, CD11c, and CD11b expression (Pichavant M et al., EBioMedecine 2015). Briefly, lung cells from air or CS-exposed mice were stained with CD11c (PE-Cy7-conjugated), F4/80 (PerCP-Cy5.5-conjugated), CD11b (V450-conjugated) and CD103 (PE-conjugated) mAbs (BioLegend). Labeled cells were isolated using a FACSAria™. Three independent populations were sorted Alveolar Macrophages (F4/80⁺ CD11c⁺), CD11b⁻ and CD11b⁺ DC (F4/80-CD11c⁺). Cell purity after sorting was consistently >98%. Post-sort analysis was performed to evaluate the expression of CD103 on DC subsets. As expected, F4/80-CD11c⁺ CD11b⁻ DC subset was CD103⁺ (97% purity) and F4/80⁻ CD11c⁺ CD11b⁺ DC subset was CD103-(98% purity).

### CD4⁺ T Cell Isolation

CD4⁺ T cells were purified from the spleen of naive animals by positive selection using CD4 microbeads (Myltenii Biotech). Isolated T cells were used for coculture with sorted APC, at a ratio 10/1. Supernatants were collected 48 hours later.

### Cytokine Quantification

Mouse IL-6, KC, IL-17, IL-22 and IFN-γ concentrations were measured in bronchoalveolar lavages (BAL), lung extracts, and supernatants of sorted APC and T cells coculture by ELISA (R&D systems).

### Reverse Transcriptase-Polymerase Chain Reaction (RT-PCR) Analysis

Quantitative RT-PCR was performed to quantify the housekeeping gene GAPDH, IL-10, RAGE, AhR, IL-6, IL-23 mRNA. Forward and reverse primers were designed as described in Table 1. Results were expressed as mean±SEM of the relative gene expression calculated for each experiment in folds (2^{-ΔΔCt}) using GAPDH as a gene reference and compared to unstimulated cells used as calibrator.

### Statistical Analysis

Results are expressed as the means±SEM. The statistical significance of differences between experimental groups was calculated by a one-way Anova with a Bonferroni post-test (GraphPad Prism® 4 Software, San Diego, CA). The possibility to use these parametric tests was assessed by checking if the population is Gaussian and the variance is equal (Bartlett's test). Results with a p value <0.05 were considered significant.

### Results

### BPZE1 Limits the Impact of Chronic Exposure to CS

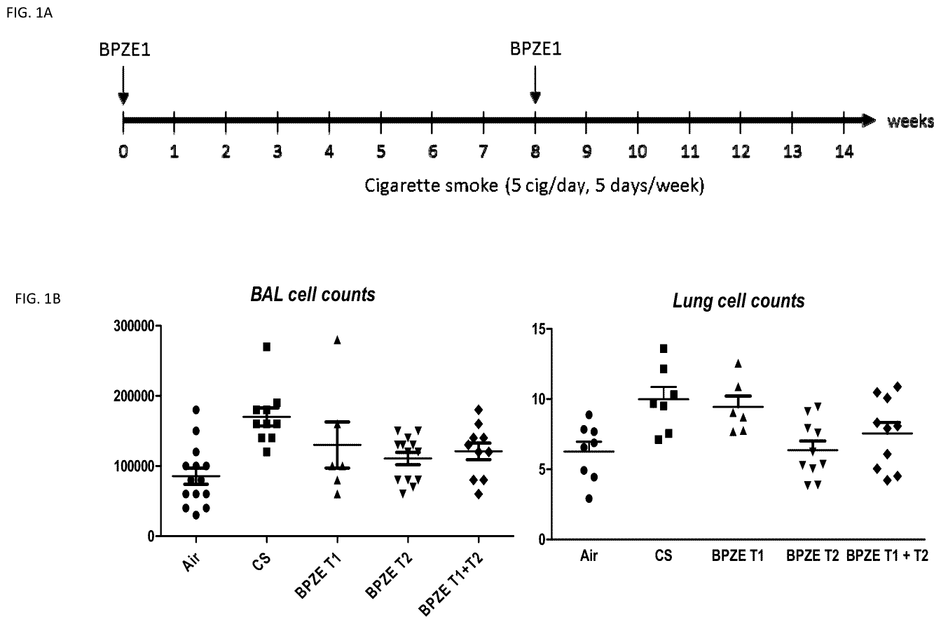

Mice were chronically exposed to cigarette smoke to develop symptoms associated with COPD, (Pichavant M et al. Mucosal Immunol 2014). To address the “off-target” effects of BPZE1 on COPD, this live attenuated vaccine was administered to mice either preventively (before chronic exposure to cigarette smoke) or curatively (in the middle of the COPD course) (FIG. 1A). As expected, chronic exposure to cigarette smoke during 12 weeks led to cellular recruitment into the BAL as well as in the lung tissues. Preventive vaccination of mice with BPZE1 limited CS-inflammation in the BAL but not in the lung tissue. In contrast, curative vaccination with BPZE1 decreased cell recruitment both in the BAL and lung tissues. Combining preventive and curative interventions did not improve the effects of BPZE1 curative treatment (FIG. 1B).

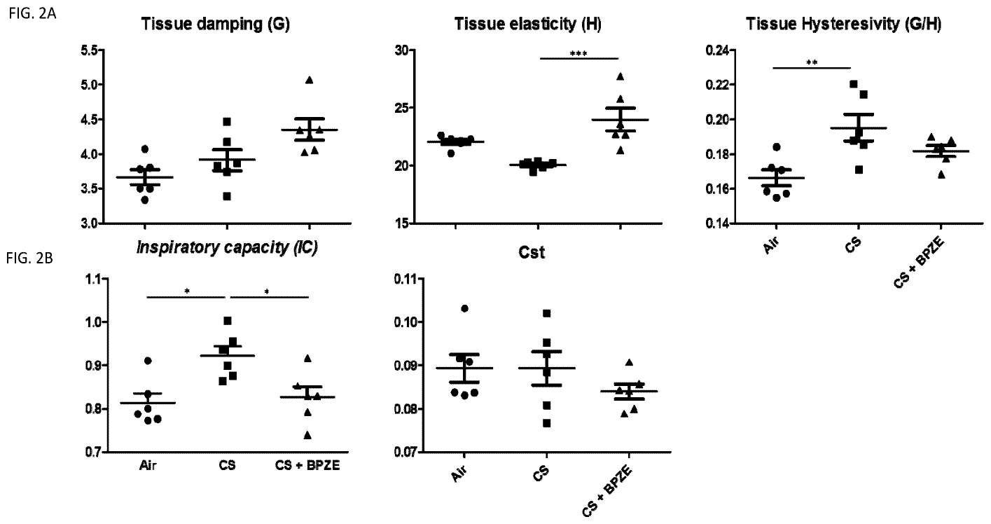

BPZE1 was able to limit the lung function decline due to CS when administered in the middle of the course of COPD development. Of note, mice vaccinated with BPZE1 before and during chronic exposure to CS did not differ much from the curatively vaccinated ones (data not shown). As depicted in FIGS. 2A and 2B, chronic exposure to CS led to decline in lung function. As expected, emphysematous mice showed statistically elevated tissue hysteresivity, increased inspiratory capacity (IC) with no change in static compliance (Cst) relative to the control mice. BPZE1 vaccination was able to partially restore lung function despite CS exposure.

### BPZE1 Vaccination Modifies the Pulmonary Immune Response to Chronic Exposure to CS

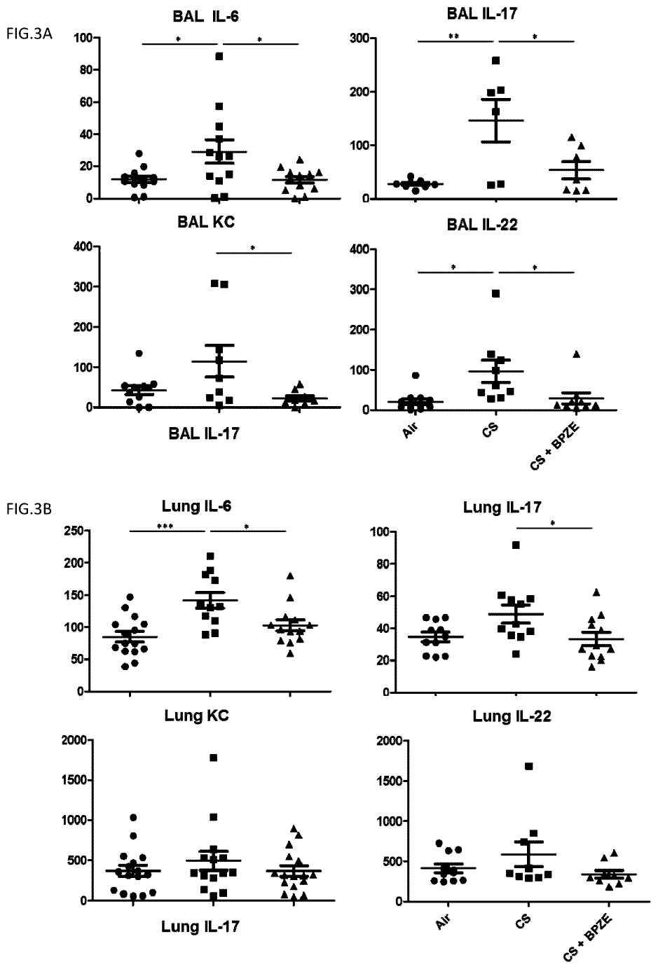

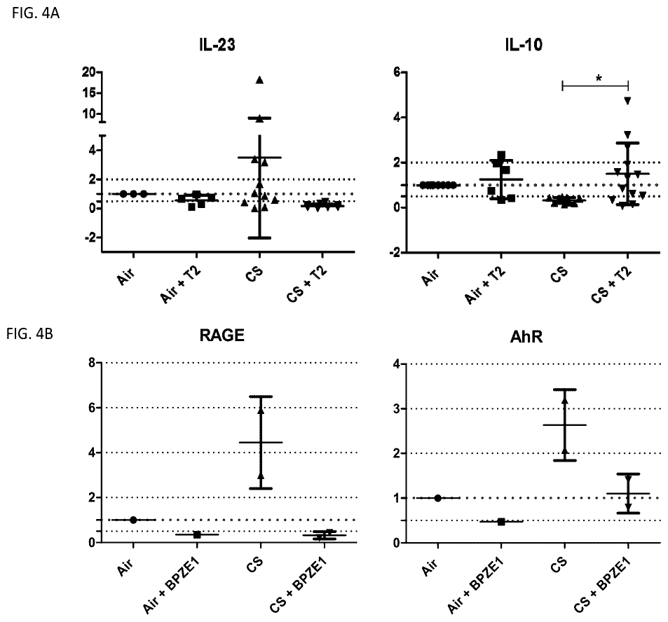

Since BPZE1 reverts the clinical outcomes of chronic exposure to CS, we examined the anti-inflammatory effects of BPZE1. We first measured cytokines and chemokines in the BAL (FIG. 3A), lung tissues (FIG. 3B), and serum (data not shown). Mice chronically exposed to CS exhibited higher levels of IL-6, KC, IL-17 and IL-22 than control Air mice, demonstrating that chronic exposure to CS led to inflammation. BPZE1 vaccination was associated to a reduction of the CS-induced inflammation. Cytokine levels were reduced in all tested compartments in BPZE1-treated COPD mice and were almost equal to baseline as seen in Air control mice. The anti-inflammatory effects of BPZE1 could be observed independently of the time of treatment (data not shown). Along the IL-17 downregulation by BPZE1, we also observed a decrease in IL-23 mRNA levels (FIG. 4A). As shown in lung tissues, the downregulation of the CS-induced inflammation by BPZE1 was associated to increased levels of the immunomodulatory IL-10 cytokine (FIG. 4A). In addition, BPZE-1 vaccination also strongly reduced Rage and AhR mRNA levels induced by chronic exposure to CS, two receptors involved in the pathophysiology of COPD (FIG. 4B).

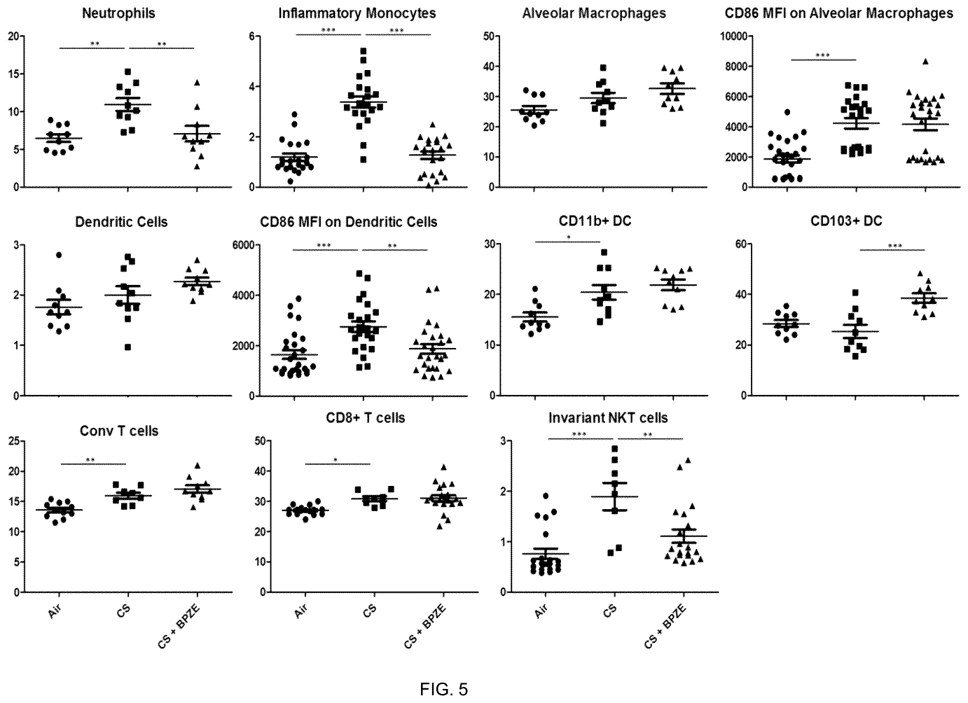

The anti-inflammatory effects of BPZE1 were also associated with a significant decrease in cellular infiltration due to CS exposure (FIG. 5). Neutrophils and CCR2+ Ly6C+ inflammatory monocytes were significantly recruited into the lungs of mice exposed to CS, and BPZE-1 vaccination significantly decreased their infiltration. Whereas BPZE1 vaccination did not impact the recruitment of APC, including alveolar macrophages and dendritic cells, BPZE1 decreased their activation due to CS exposure, as depicted by the decrease expression of CD86. Only the subpopulation CD103+ of dendritic cells were recruited into the lung tissues after BPZE1 vaccination, a subpopulation known to play tolerogenic roles. Conventional T cells were not impacted by the treatment, but the recruitment of innate immune cells like NKT cells due to CS exposure were downregulated by BPZE1 vaccination.

Thus, BPZE1 appears to exert anti-inflammatory effects on COPD, by surprisingly limiting the inflammatory Th17 cytokine production and by modulating innate as well as adaptive immune cells in response to CS.

### BPZE1 Vaccination Limits Th17 Response and Induces Tolerogenic Antigen Presenting Cells

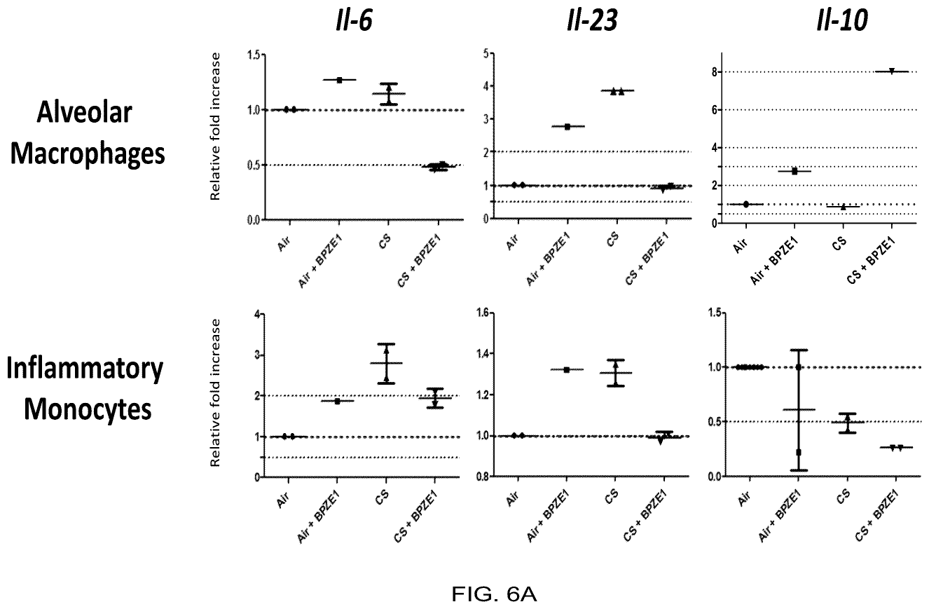

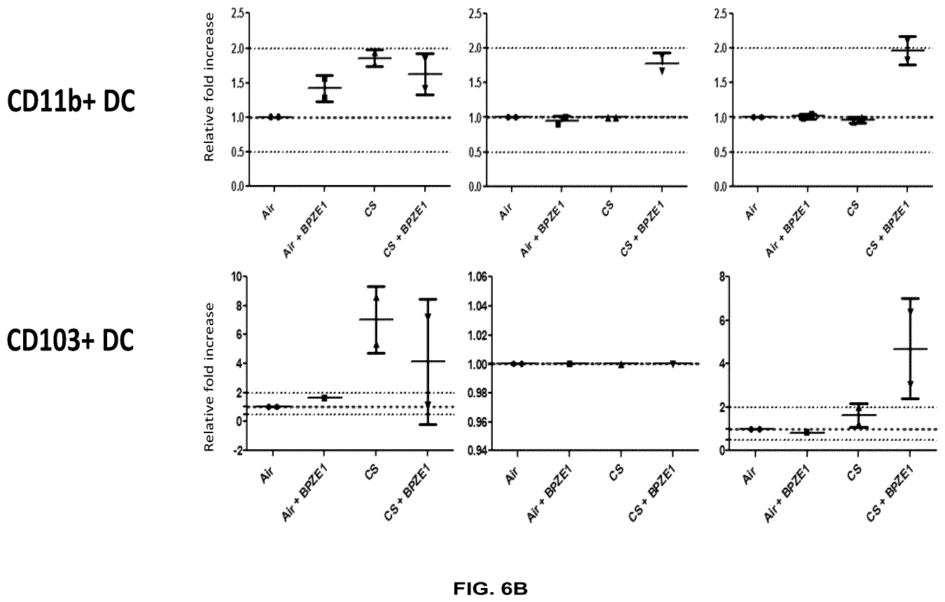

Since BPZE1 vaccination surprisingly limited IL-17 and IL-22 levels (FIGS. 3A and 3B), we focused on pro-Th17 factors, including IL-6 and IL-23 in antigen-presenting cells. As depicted in FIG. 6, we performed cell sorting on lung tissues 2 weeks after BPZE1 vaccination, leading to the isolation of 4 populations: alveolar macrophages, inflammatory monocytes, CD11b+ dendritic cells and CD103+ dendritic cells. We first evaluated the cytokine profile of these sorted cell populations. BPZE1 limited the expression of il-6 and il-23 mRNA induced by CS exposure in sorted alveolar macrophages and inflammatory monocytes. In contrast, no change was observed in sorted dendritic cells.

We also observed that BPZE-1 vaccination led to increased IL-10 mRNA levels in CS-exposed mice (FIG. 4A). We therefore examined IL-10 mRNA levels in sorted pulmonary antigen-presenting cells. BPZE-1 vaccination led to increased Il-10 mRNA levels in alveolar macrophages, and CD11b⁺ and CD103⁺ dendritic cells of COPD mice.

### CONCLUSION

As a conclusion, BPZE-1 vaccination is able to limit COPD outcomes in mice, by decreasing the CS-induced inflammation and restoring partially the CS-induced lung function alteration. This could be surprisingly explained by the decrease of the Th17 pathway and the induction of an IL-10 response in the context of CS exposure. These results are in opposition to the teaching of the prior art wherein it was previously shown that BPZE1 rather drives a Th17 response (Schiavoni I, Fedele G, Quattrini A, Bianco M, Schnoeller C, Openshaw P J, Locht C, Ausiello C M. Live attenuated B. pertussis BPZE1 rescues the immune functions of Respiratory Syncytial virus infected human dendritic cells by promoting Th1/Th17 responses. PLoS One. 2014 Jun. 26; 9 (6):e100166). (Schnoeller C, Roux X, Sawant D, Raze D, Olszewska W, Locht C, Openshaw P J. Attenuated Bordetella pertussis vaccine protects against respiratory syncytial virus disease via an IL-17-dependent mechanism. Am J Respir Crit Care Med. 2014 January; 189 (2): 194-202).

What is claimed is:

1. A method of treating chronic obstructive pulmonary disease in a patient in need thereof comprising administering a therapeutically effective amount of a mutated Bordetella strain, wherein the strain comprises a mutated pertussis toxin (ptx) gene, a deleted or mutated dermonecrotic (dnt) gene, and a heterologous ampG gene.

2. The method of claim 1 wherein the patient suffers from a moderate, a severe or a very severe COPD.

3. The method of claim 1 is suitable for preventing progression of COPD in a patient.

4. The method of claim 1 for preventing progression of COPD in a patient from one stage to a subsequent stage in the GOLD classification.

5. The method of claim 1 wherein, the Bordetella starting strain which is mutated can be any Bordetella strain including Bordetella pertussis, Bordetella parapertussis, and Bordetella bronchiseptica.

6. The method of claim 1 wherein the starting strain used to obtain the mutated Bordetella strain is Bordetella pertussis.

7. The method of claim 6 wherein the mutated strain is BPZE1 strain that is identified by the accession number CNCM I-3585.

8. The method of claim 1 wherein additional mutations are introduced, such as pertactin deficient mutants, adenylate cyclase (AC) deficient mutants, filamentous hemagglutinin (FHA), and any of the bvg-regulated components.

9. The method of claim 8 wherein the Bordetella strain is also deficient for pertactin.

10. The method of claim 1 wherein the mutated Bordetella strain is attenuated.

11. The method of claim 1 wherein the mutated Bordetella strain is administered to the patient as live vaccine.

12. The method of claim 1 wherein the mutated Bordetella strain is administered to the patient by nasal administration.

13. The method of claim 12 wherein the mutated Bordetella strain is administered to the patient by an aerosol.

14. The method of claim 1 wherein the mutated Bordetella strain is administered to the patient in combination with at one adjuvant.
